(19) United States
(12) Patent Application Publication
(10) Pub. No.: US 2015/0068527 A1
(43) Pub. Date: Mar. 12, 2015

(54) TURBINE VENTILATOR PRESSURE-CONTROLLED VENTILATION METHOD

(71) Applicant: BEIJING AEONMED CO., LTD., Beijing (CN)

(72) Inventor: Jie Cheng, Beijing (CN)

(21) Appl. No.: 14/395,292

(22) PCT Filed: Oct. 22, 2013
(86) PCT No.: PCT/CN2013/085723

§ 371 Date: Oct. 17, 2014

(30) Foreign Application Priority Data
Dec. 26, 2012 (CN) 201210575970.5

(51) Int. Cl. A61M 16/00; A61M 16/20

(52) U.S. Cl. CPC A61M 16/0069; A61M 16/0003; A61M 16/204; A61M 16/205; A61M 2016/0027; A61M 2016/0015; A61M 2205/3331
USPC 128/204.23

(57) ABSTRACT

A turbine ventilator pressure-controlled ventilation method comprising the following steps: a ventilator is started up, a control unit in the ventilator controls a turbine motor to rotate at rotational speed U, the turbine motor provides the ventilator with a hyperbaric gas; a detector unit detects the breathing state of a patient, if the patient is in an inhalation state, proceeds to an inhalation phase control, and, if the patient is in an exhalation state, proceeds to an exhalation phase control; the air pressure of an inhalation phase is controlled by the control unit by controlling driving voltage V₁ of an inhalation valve to regulate the extent to which the inhalation valve is opened, the positive end-expiratory pressure of an exhalation phase is controlled by the control unit by controlling driving voltage V₂ of an exhalation valve to regulate the extent to which the exhalation valve is opened.

### TECHNICAL FIELD

The present application relates to the field of control technologies for ventilator ventilation pressure, and in particular to a pressure-controlled ventilation method for a turbine ventilator.

### BACKGROUND

Currently, volume control or pressure control is mostly employed in controlling an anesthesia machine and a ventilator. In general, either of the volume control and the pressure control can be applied to merely a group of special patients. The pressure control is advantageous in that a patient can be supplied regularly with a gas at a specified pressure according to a pressure set by a doctor, with the gas being supplied each time at almost an identical pressure, so that the pressure control can be applied to a big group of patients, including patients suffering from a lung lesion, infants and children.

Among ventilator ventilation modes, the pressure-controlled ventilation mode is the most basic. In a conventional ventilator, an air supply is a high-pressure gas provided by an air compressor or an external device, thus the control of the pressure-controlled ventilation (PCV) is implemented by controlling an opening degree of an inspiratory valve, and the value of a target pressure is monitored in real time based on a feedback from a pressure sensor. However, in a turbine ventilator, the air supply is a high-pressure gas generated by rotation of the turbine, thus the PCV involves not only controlling the target pressure but also computing a rotation speed of the turbine in the turbine ventilator. An excessively low rotation speed of the turbine may cause that the target pressure cannot be reached, and an excessively high rotation speed of the turbine may cause that the target pressure is out of control, and hence cause a damage risk.

### SUMMARY

Embodiments of the present disclosure provide a pressure-controlled ventilation method for a turbine ventilator, which can accurately control a rotation speed of a motor and a target pressure, so that the turbine ventilator has high safety, stability and reliability.

To this end, the technical solution of the present disclosure is provided below.

A pressure-controlled ventilation method for a turbine ventilator includes Steps A to E below:

Step A of starting up a ventilator, wherein a control unit of the ventilator controls a turbine motor to rotate at a rotation speed U, and the turbine motor is configured for providing the ventilator with a high-pressure gas;

Step B of detecting a breath state of a patient by a detection unit, wherein if the patient is in an inspiration state, Step C is performed, otherwise, if the patient is in an expiration state, Step D is performed;

Step C of adjusting an opening degree of an inspiratory valve by controlling a driving voltage V₁ for the inspiratory valve by a control unit, to control air pressure in an inspiration phase, and performing Step D or Step E after the inspiration phase control ends;

Step D of adjusting an opening degree of an expiratory valve by controlling a driving voltage V₂ for the expiratory valve by the control unit, to control positive end-expiratory pressure in an expiration phase, and performing Step C or Step E after the expiration phase control ends; and

Step E of ending auxiliary air supply from the ventilator to the patient and shutting down the ventilator.

Preferably, the rotation speed U of the turbine motor is calculated by a formula of:

U=R_{—}VCV*Qt arg et+Ti*Qt arg et/C_{—}VCV+PEEP_Set,

wherein, R_VCV denotes system resistance, Qtarget denotes a preset flow velocity, Ti denotes inspiration time, C_VCV denotes system compliance, and PEEP_Set denotes a preset positive end-expiratory pressure value.

Preferably, the preset flow velocity Qtarget is calculated by a formula of:

Qt arg et=TV/T,

wherein, R_VCV denotes a feedback value of tidal volume, i.e. a total inspiratory tidal volume in an immediately previous period, and T denotes inspiration time.

Preferably, the control unit is configured to calculate the required rotation speed U of the motor through the formula of calculating the rotation speed of the turbine motor according to a preset tidal volume value, the preset positive post-expiratory pressure value, the inspiration time and the preset flow velocity which are read by a read unit, and control the motor to rotate at the rotation speed U.

Preferably, in Step C, the driving voltage V₁ for the inspiratory valve is calculated by formulas of:

feedforward_Ctrl=K₁*Pset+B₁,

V₁=feedforward_Ctrl+kp_{—}P*(P_set−lp_{—}P)+kd_{—}P*(0−(lp_{—}P−last_{—}lp_{—}P)),

wherein, Pset denotes a preset pressure value, K₁ and B₁ denote proportionality coefficients, feedforward_Ctrl denotes a feedforward voltage, i.e. a voltage required for the inspiratory valve under a preset pressure, kp_p denotes a proportionality coefficient, P_set denotes a preset pressure value, lp_P denotes a pressure feedback value, kd_P denotes a differential coefficient of a proportional-integral-derivative (PID) controller, and last_lp_P denotes a previous pressure feedback value.

Preferably, the proportionality coefficients K₁ and B₁ depend on characteristics of the inspiratory valve, and values of K₁ and B₁ are determined from a pressure-voltage curve obtained from a plurality of calibrations for the inspiratory valve.

Preferably, in Step D, the driving voltage V₂ for the expiratory valve is calculated by a formula of:

V₂=k₂*(Peep+DP)+B₂,

where, Peep denotes positive end-expiratory pressure, DP denotes a difference between the preset positive end-expiratory pressure value and a monitored positive end-expiratory pressure value, and K₂ and B₂ are coefficients.

Preferably, the proportionality coefficients K₂ and B₂ depend on characteristics of the expiratory valve, and values of K₂ and B₂ are determined from a pressure-voltage curve obtained from a plurality of calibrations for the expiratory valve.

Preferably, in Step C, if pressure detected by a pressure sensor exceeds an upper limit for an alarm, or exceeds the target pressure by 3 centimeters of water, or inspiration time has expired, then the control unit controls the ventilator to switch from inspiration to expiration.

Preferably, in Step D, if expiration time expires or a patient trigger occurs, then the control unit controls the ventilator to switch from expiration to inspiration.

The beneficial effects of the present disclosure lie that: in the pressure-controlled ventilation method for the turbine ventilator provided in the present disclosure, the operation parameters of the ventilator such as the system resistance R_VCV, the system compliance C_VCV, and the set Positive End-Expiratory Pressure (PEEP) value PEEP_Set are combined with control of the turbine speed, in order to achieve a constant flow under the control of the turbine and real-time synchronous control, that is, an input voltage of the inspiratory valve and an input voltage of the expiratory valve in the ventilator are controlled in real time in order to achieve accurate control of the rotation speed of the motor and the target pressure, so that the turbine ventilator has high safety, stability and reliability.

### DESCRIPTION OF DRAWINGS

FIG. 1 is a flowchart showing a pressure-controlled ventilation method for a turbine ventilator according to an embodiment of the present invention;

FIG. 2 is a flowchart showing an inspiration control in the pressure-controlled ventilation method for the turbine ventilator according to an embodiment of the present invention; and

FIG. 3 is a flowchart showing an expiration control in the pressure-controlled ventilation method for the turbine ventilator according to an embodiment of the present invention.

### DETAILED DESCRIPTION OF THE EMBODIMENT

Technical solutions of the present invention are further described below by specific embodiments in conjunction with the accompanying drawings.

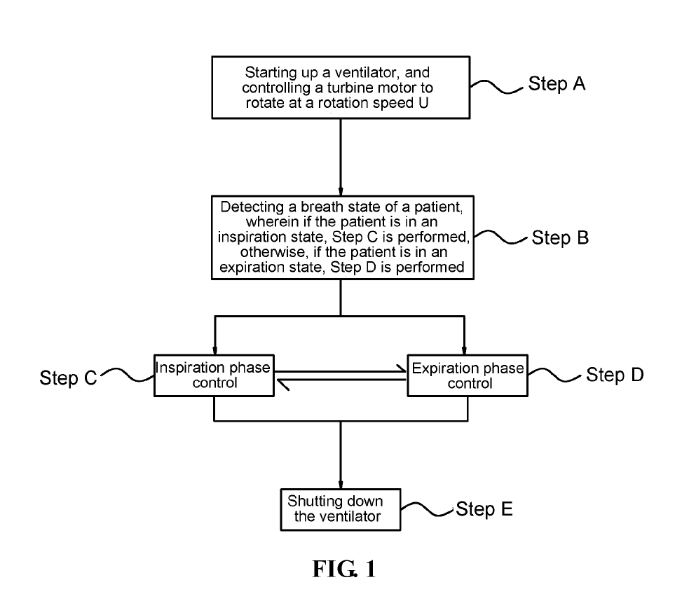

As shown in FIG. 1, a pressure-controlled ventilation method for a turbine ventilator includes Steps A to E below:

at Step A: starting up a ventilator, wherein a control unit of the ventilator controls a turbine motor to rotate at a rotation speed U, and the turbine motor is configured for providing the ventilator with a high-pressure gas;

at Step B: detecting a breath state of a patient by a detection unit, wherein if the patient is in an inspiration state, Step C is performed to perform inspiration phase control on the ventilator, otherwise, if the patient is in an expiration state, Step D is performed to perform expiration phase control on the patient;

at Step C: adjusting an opening degree of an inspiratory valve by controlling a driving voltage V₁ for the inspiratory valve by a control unit, to control air pressure in an inspiration phase, and performing Step D or Step E after the inspiration phase control ends;

at Step D: adjusting an opening degree of an expiratory valve by controlling a driving voltage V₂ for the expiratory valve by the control unit, to control positive end-expiratory pressure in an expiration phase, and performing Step C or Step E after the expiration phase control ends; and

at Step E: ending auxiliary air supply from the ventilator to the patient and shutting down the ventilator.

In Step A, in the turbine control system, since the turbine has low responsivity and hence is not suitable for real-time control, a constant voltage is applied to the turbine during the inspiration control and the expiration control in the ventilation process, so that the rotation speed of the turbine is maintained constant. The size of the rotation speed of the turbine depends on the system resistance, the system compliance and the preset tidal volume, and thus a rotation speed U of a turbine motor (i.e. a motor for the turbine) is calculated by a formula of:

U=R_{—}VCV*Qt arg et+Ti*Qt arg et/C_{—}VCV+PEEP_Set,

where, R_VCV denotes system resistance; Qtarget denotes a preset flow velocity; Ti denotes inspiration time; C_VCV denotes system compliance; and PEEP_Set denotes a preset positive end-expiratory pressure (PEEP) value.

The preset flow velocity is equal to the tidal volume divided by the inspiration time, and thus the preset flow velocity Qtarget is calculated by a formula of:

Qt arg et=TV/T,

where, TV denotes a feedback value of tidal volume, i.e. a total inspiratory tidal volume in an immediately previous period, and T denotes inspiration time.

The control unit of the ventilator is configured to calculate the required rotation speed U of the motor through the above formula of calculating the rotation speed of the turbine motor according to a total inspiratory tidal volume in an immediately previous period, the preset positive post-expiratory pressure value, the inspiration time and the preset flow velocity which are read by a read unit, and control the motor to rotate at the rotation speed U.

The PCV control mainly includes an inspiration phase control and an expiration phase control. In the inspiration phase control, the control object of the inspiration phase control is a preset pressure value Pset, which is specifically implemented by controlling the opening degree of the inspiratory valve. The opening degree of the inspiratory valve is determined by the driving voltage provided with the inspiratory valve, and in Step C, the driving voltage V₁ for the inspiratory valve is calculated by formulas of:

feedforward_Ctrl=K₁*Pset+B₁,

V₁=feedforward_Ctrl+kp_{—}P*(P_set−lp_{—}P)+kd_{—}P*(0−(lp_{—}P−last_{—}lp_{—}P)),

where, Pset denotes a preset pressure value, K₁ and B₁ denote proportionality coefficients, feedforward_Ctrl denotes a feedforward voltage, i.e. a voltage required for the inspiratory valve under a preset pressure, kp_p denotes a proportionality coefficient, P_set denotes a preset pressure value, lp_P denotes a pressure feedback value, kd_P is a differential coefficient of the a PID controller, and last_lp_P denotes a previous pressure feedback value.

The proportionality coefficients K₁ and B₁ depend on characteristics of the inspiratory valve, and values of K₁ and B₁ are determined from a pressure-voltage curve obtained from a plurality of calibrations for the inspiratory valve. The inaccurate calibration for the values of K₂ and B₂ would cause inaccurate control of the target pressure.

In the process of the expiration phase control, if pressure detected by a pressure sensor exceeds an upper limit for an alarm, or exceeds the target pressure by 3 centimeters of water, or inspiration time has expired, then the control unit controls the ventilator to switch from inspiration to expiration.

In the process of the expiration phase control, the control object of the expiration phase control is a preset PEEP, i.e. the positive end-expiratory pressure value, which is specifically implemented by the opening degree of the expiratory valve. The opening degree of the expiratory valve is determined by the driving voltage provided with the expiratory valve, and in Step D, the driving voltage V₂ for the expiratory valve is calculated by a formula of:

V₂=k₂*(Peep+DP)+B₂,

where, Peep is the positive end-expiratory pressure, DP is a difference between the preset PEEP value and the monitored PEEP value, K₂ and B₂ are coefficients.

The proportionality coefficients K₂ and B₂ depend on characteristics of the expiratory valve, and values of K₂ and B₂ are determined from a pressure-voltage curve obtained from a plurality of calibrations for the expiratory valve. The inaccurate calibration for the values of K₂ and B₂ would cause inaccurate PEEP control.

Closed-loop PEEP regulation is further added in the process of the expiration phase control. If the PEEP in the immediately previous period is too high, the value of DP, i.e. the preset PEEP value minus the monitored PEEP value, is less than zero, and if the PEEP in the immediately previous period is too low, the value of DP, i.e. the preset PEEP value minus the monitored PEEP value, is larger than zero, thereby improving the accuracy of controlling the expiratory valve.

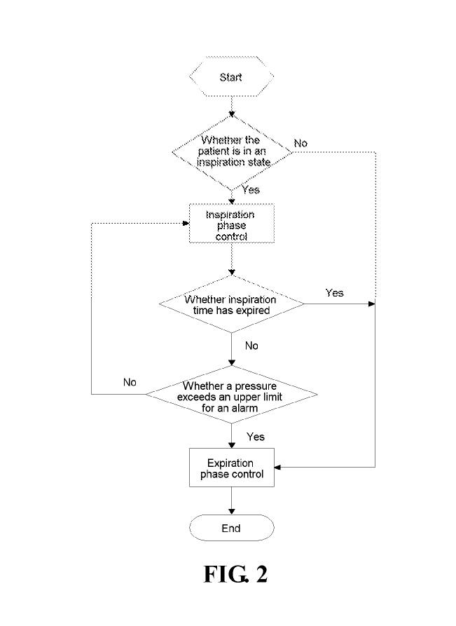

FIG. 2 is a flowchart showing an inspiration control in the pressure-controlled ventilation method for the turbine ventilator according to an embodiment of the present invention. As shown, the detection unit detects a breath state of a patient, and if the patient attempts to inspire, then the inspiration phase control starts whilst the control unit detects in real time a pressure value of the breath loop by the pressure sensor connected with the control unit. If the pressure detected by the pressure sensor exceeds an upper limit for an alarm or exceeds the target pressure by 3 centimeters of water (cmH₂O), or the preset inspiration time has expired, then the control unit controls the ventilator to switch from inspiration to expiration, thus the inspiration phase control ends and the expiration phase control starts. Additionally, if the air supplying to the patient need be stopped in the inspiration phase control, the ventilator is shut down.

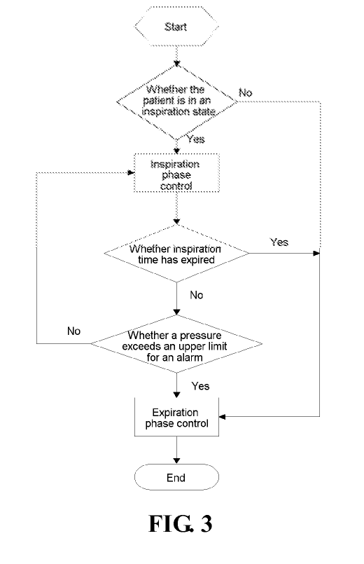

FIG. 3 is a flowchart showing expiration control in the pressure-controlled ventilation method for the turbine ventilator according to an embodiment of the present invention. As shown, the detection unit detects a breath state of a patient, and if the patient attempts to expire, then the expiration phase control starts. During the expiration phase control, it is detected in real time whether the expiration time has expired. If the expiration time has expired, then the ventilator is switched from the expiration phase control to the inspiration phase control. While monitoring the time, it is also detected in real time whether a patient trigger occurs, and if the patient trigger occurs, then the ventilator need also switch to the expiration phase control. Additionally, after the expiration phase control ends, if the air supplying to the patient need be stopped, the ventilator is shut down directly.

The technical principle of the present disclosure has described as above by combining the specific embodiments, which are merely intended to explain the principle of the present disclosure, but cannot be interpreted in any manner as limitation to the present disclosure. In light of the explanation herein, other embodiments of the present disclosure conceived by those skilled in the art without any creative work should fall into the scope of protection of the present invention.

1. A pressure-controlled ventilation method for a turbine ventilator, comprising:
Step A of starting up a ventilator, wherein a control unit of the ventilator controls a turbine motor to rotate at a rotation speed U, and the turbine motor is configured for providing the ventilator with a high-pressure gas;
Step B of detecting a breath state of a patient by a detection unit, wherein if the patient is in an inspiration state, Step C is performed to perform inspiration phase control on the ventilator, otherwise, if the patient is in an expiration state, Step D is performed to perform expiration phase control on the patient;
Step C of adjusting an opening degree of an inspiratory valve by controlling a driving voltage V₁ for the inspiratory valve by a control unit, to control air pressure in an inspiration phase, and performing Step D or Step E after the inspiration phase control ends;
Step D of adjusting an opening degree of an expiratory valve by controlling a driving voltage V₂ for the expiratory valve by the control unit, to control positive end-expiratory pressure in an expiration phase, and performing Step C or Step E after the expiration phase control ends; and
Step E of ending auxiliary air supply from the ventilator to the patient and shutting down the ventilator.

2. The pressure-controlled ventilation method of claim 1, wherein, the rotation speed U of the turbine motor is calculated by a formula of:
U=R_{—}VCV*Qt arg et+Ti*Qt arg et/C_{—}VCV+PEEP_Set,
wherein, R_VCV denotes system resistance, Qtarget denotes a preset flow velocity, Ti denotes inspiration time, C_VCV denotes system compliance, and PEEP_Set denotes a preset positive end-expiratory pressure value.

3. The pressure-controlled ventilation method of claim 2, wherein, the preset flow velocity Qtarget is calculated by a formula of:
Qt arg et=TV/T,
wherein, TV denotes a feedback value of tidal volume, i.e. a total inspiratory tidal volume in an immediately previous period, and T denotes inspiration time.

4. The pressure-controlled ventilation method of claim 3, wherein, the control unit is configured to calculate the required rotation speed U of the motor through the formula of calculating the rotation speed of the turbine motor according to a preset tidal volume value, the preset positive post-expiratory pressure value, the inspiration time and the preset flow velocity which are read by a read unit, and control the motor to rotate at the rotation speed U.

5. The pressure-controlled ventilation method of claim 1, wherein, in Step C, the driving voltage V₁ for the inspiratory valve is calculated by formulas of:
feedforward_Ctrl=K₁*Pset+B₁,
V₁=feedforward_Ctrl+kp_{—}P*(P_set−lp_{—}P)+kd_{—}P*(0−(lp_{—}P−last_{—}lp_{—}P)),
wherein, Pset denotes a preset pressure value, K₁ and B₁ denote proportionality coefficients, feedforward_Ctrl denotes a feedforward voltage, i.e. a voltage required for the inspiratory valve under a preset pressure, kp_p denotes a proportionality coefficient, P_set denotes a preset pressure value, lp_P denotes a pressure feedback value, kd_P denotes a differential coefficient of a proportional-integral-derivative (PID) controller, and last_lp_P denotes a previous pressure feedback value.

6. The pressure-controlled ventilation method of claim 5, wherein, the proportionality coefficients K₁ and B₁ depend on characteristics of the inspiratory valve, and values of K₁ and B₁ are determined from a pressure-voltage curve obtained from a plurality of calibrations for the inspiratory valve.

7. The pressure-controlled ventilation method of claim 1, wherein, in Step D, the driving voltage V₂ for the expiratory valve is calculated by a formula of:
V₂=k₂*(Peep+DP)+B₂,
wherein, Peep denotes positive end-expiratory pressure, DP denotes a difference between the preset positive end-expiratory pressure value and a monitored positive end-expiratory pressure value, and K₂ and B₂ are coefficients.

8. The pressure-controlled ventilation method of claim 7, wherein, the proportionality coefficients K₂ and B₂ depend on characteristics of the expiratory valve, and values of K₂ and B₂ are determined from a pressure-voltage curve obtained from a plurality of calibrations for the expiratory valve.

9. The pressure-controlled ventilation method of claim 1, wherein,
in Step C, if pressure detected by a pressure sensor exceeds an upper limit for an alarm, or exceeds the target pressure by 3 centimeters of water, or inspiration time has expired, then the control unit controls the ventilator to switch from inspiration to expiration.

10. The pressure-controlled ventilation method of claim 1, wherein, in Step D, if expiration time expires or a patient trigger occurs, then the control unit controls the ventilator to switch from expiration to inspiration.
